(19) United States
(12) Patent Application Publication
(10) Pub. No.: US 2026/0262957 A1
(43) Pub. Date: Sep. 10, 2026

(54) APPARATUS AND METHOD FOR DETERMINING BLOOD VELOCITY

(71) Applicant: Japan Display Inc., Tokyo (JP)

(72) Inventor: Adithya NARESH, Hayward, CA (US)

(73) Assignee: Japan Display Inc., Tokyo (JP)

(21) Appl. No.: 19/070,587

(22) Filed: Mar. 5, 2025

(51) Int. Cl. A61B 5/0285; A61B 5/026; G06T 7/00; G06T 7/246

(52) U.S. Cl. CPC A61B 5/0285; A61B 5/0261; G06T 7/0012; G06T 7/246; G06T 2207/10048; G06T 2207/30104

(57) ABSTRACT

A blood velocity measurement apparatus includes a near-infrared (NIR) light source configured to irradiate a subject with NIR light, and a NIR sensor configured to receive scattered NIR light from the subject and produce a series of NIR image frames. Each NIR image frame in the series of NIR image frames represents a two dimensional image of a same region of the subject irradiated by the NIR light source and at a different time. The apparatus also includes processing circuitry configured to calculate a blood velocity value from the series of NIR image frames. A corresponding method determines the blood velocity value.

### FIELD OF THE INVENTION

The present disclosure relates generally to an apparatus and method for determination of blood velocity, and in particular, for the measurement of blood velocity by extracting blood velocity biomarker measurements from near infrared image sensors.

### BACKGROUND OF INVENTION

Biomarkers are quantitative metrics computed from various types of sensor measurements of the human body with correlation to the body's physiological signals and conditions (examples: heart rate, respiration rate, etc.). As shown in FIG. 1, certain common biomarkers may be easily computed and quantified using inexpensive, readily available sensor technologies. As shown by FIG. 2 there are less-common biomarkers, including the blood velocity biomarker, that may not be quantifiable using currently available optical sensing technologies.

The blood velocity biomarker represents the speed of blood at a particular point (i.e., location) typically along an artery/vein. The blood velocity biomarker can have several uses for a medical practitioner, which may include tracking localized issues that correlate with a change in blood velocity. For example, for someone with a chronic condition like carpel tunnel syndrome, a change in blood velocity trends over time in the wrist area can indicate progression or regression. Similarly, for someone with chronic migraines, blood velocity trends on the forehead temple region can be used for tracking migraine incidents.

Conventionally, blood velocity is measured using expensive and bulky technologies including doppler ultrasound and magnetic resonance imaging equipment. Doppler ultrasound equipment, for example, sends ultrasound frequency mechanical waves into the skin causing the waves to echo back to the ultrasound sensor array in the device. When the waves encounter moving blood within a vessel, like an artery or vein, the waves reflect with a phase shift proportional to the speed of the moving blood. The phase shift may be imaged along with the various layers of the skin, muscle, and soft tissue, and the image is used to compute the blood velocity. Conventional technology has various limitations including bulkiness, excessive cost, low resolution and slow framerate.

### SUMMARY OF INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

An embodiment of the invention includes a blood velocity measurement apparatus comprising: a near-infrared (NIR) light source configured to irradiate a subject with NIR light; a NIR sensor configured to receive scattered NIR light from the subject and produce a series of NIR image frames, each NIR image frame in the series of NIR image frames representing a two dimensional image of a same region of the subject irradiated by the NIR light source and at a different time; and processing circuitry configured to calculate a blood velocity value from the series of NIR image frames.

Another embodiment of the invention includes the blood velocity measurement apparatus, wherein the blood velocity value includes at least one of an average blood flow velocity and an instantaneous blood flow velocity.

Another embodiment of the invention includes the blood velocity measurement apparatus, wherein the processing circuitry is further configured to identify one or more blood flow clusters within the series of NIR image frames; and calculate the blood velocity value based on movement the one or more blood flow clusters between frames of the series of NIR image frames.

Another embodiment of the invention includes the blood velocity measurement apparatus, wherein the processing circuitry is further configured to: identify one or more blood flow clusters within the series of NIR image frames; identify only one of the one or more blood flow clusters as a key blood flow cluster; determine a center of mass of the key blood flow cluster in the series of NIR image frames; determine a movement of the key blood flow cluster based on the center of mass of the key blood flow cluster in the series of NIR image frames; and determine the blood velocity value from the determined movement of the key blood flow cluster in the series of NIR image frames.

Another embodiment of the invention includes the blood velocity measurement apparatus, wherein the processing circuitry is further configured to: identify one or more regions of higher intensity pixels in each frame of the series of NIR image frames; calculate at least one property of each of the regions of higher intensity pixels; and identify as the one or more blood flow clusters each region of higher intensity pixels for which the at least one property meets a predetermined requirement.

Another embodiment of the invention includes the blood velocity measurement apparatus, wherein the processing circuitry is further configured to determine at least one of the following as the at least one property: a center of mass pixel coordinate of a corresponding region of higher intensity pixels key blood flow, a width of a corresponding region of higher intensity pixels, a length of a corresponding region of higher intensity pixels, and an area of a corresponding region of higher intensity pixels.

Another embodiment of the invention includes the blood velocity measurement apparatus, wherein the processing circuitry is further configured to identify one of the one or more blood flow clusters as the key blood flow cluster based on a degree of similarity of a movement of each blood flow cluster as compared to a cardiac rate of the subject; and a degree of consistency in a shape of each blood flow cluster from frame to frame in the series of NIR image frames.

Another embodiment of the invention includes the blood velocity measurement apparatus, wherein the processing circuitry is further configured to determine the movement of the key blood flow cluster based on a position of a center of mass of the key blood flow cluster in each of a plurality of frames of the series of NIR image frames.

Another embodiment of the invention includes the blood velocity measurement apparatus, wherein at least one of the NIR light source and the NIR sensor is not in direct contact with the subject.

Another embodiment of the invention includes the blood velocity measurement apparatus, wherein at least one of the NIR light source and the NIR sensor is in direct contact with the subject.

Another embodiment of the invention includes the blood velocity measurement apparatus, wherein the NIR light source and the NIR sensor are provided on a same substrate.

Another embodiment of the invention includes a method of measuring blood velocity in a subject, the method comprising: irradiating a subject with NIR light; receiving scattered NIR light from the subject; producing a series of NIR image frames, each NIR image frame in the series of NIR image frames representing a two dimensional image of a same region of the subject irradiated by the NIR light and at a different time; and calculating a blood velocity value from the series of NIR image frames.

Another embodiment of the invention includes the method, wherein the blood velocity value includes at least one of an average blood flow velocity and an instantaneous blood flow velocity.

Another embodiment of the invention includes the method, further comprising: identifying one or more blood flow clusters within the series of NIR image frames; and calculating the blood velocity value based on movement the one or more blood flow clusters between frames of the series of NIR image frames.

Another embodiment of the invention includes the method, further comprising: identifying one or more blood flow clusters within the series of NIR image frames; identifying only one of the one or more blood flow clusters as a key blood flow cluster; determining at least one property of the key blood flow cluster in the series of NIR image frames; determining a movement of the key blood flow cluster based on the at least one property of the key blood flow cluster in the series of NIR image frames; and determining the blood velocity value from the determined movement of the key blood flow cluster in the series of NIR image frames.

Another embodiment of the invention includes the method, further comprising: identifying one or more regions of higher intensity pixels in each frame of the series of NIR image frames; calculating at least one property of each of the regions of higher intensity pixels; and identifying as the one or more blood flow clusters each region of higher intensity pixels for which the at least one property meets a predetermined requirement.

Another embodiment of the invention includes the method, further comprising: determining at least one of the following as the at least one property a center of mass pixel coordinate of a corresponding region of higher intensity pixels key blood flow, a width of a corresponding region of higher intensity pixels, a length of a corresponding region of higher intensity pixels, and an area of a corresponding region of higher intensity pixels.

Another embodiment of the invention includes the method, further comprising: identifying one of the one or more blood flow clusters as the key blood flow cluster based on a degree of similarity of a movement of each blood flow cluster as compared to a cardiac rate of the subject and a degree of consistency in a shape of each blood flow cluster from frame to frame in the series of NIR image frames.

Another embodiment of the invention includes the method, further comprising: determining the movement of the key blood flow cluster based on a position of a center of mass of the key blood flow cluster in each of a plurality of frames of the series of NIR image frames.

Another embodiment of the invention includes the method, further comprising: diagnosing a condition of the subject based on the blood velocity value.

### BRIEF DESCRIPTION OF THE DRAWINGS

The scope of the present disclosure is best understood from the following detailed description of exemplary embodiments when read in conjunction with the accompanying drawings, wherein:

FIG. 1 lists some common biomarkers;

FIG. 2 lists some less common biomarkers;

FIG. 3A shows a candidate imaged area of a subject according to an embodiment of the invention;

FIG. 3B shows another view of the candidate imaged area;

FIG. 3C shows a digitized NIR sensed image series according to an embodiment of the invention;

FIG. 4A is an example of an apparatus according to an embodiment of the invention, using a non-contact arrangement;

FIG. 4B is another example of an apparatus according to an embodiment of the invention using a contact arrangement;

FIG. 4C is another example of an apparatus according to an embodiment of the invention using a contact arrangement;

FIG. 4D is another example of an apparatus according to an embodiment of the invention using a non-contact arrangement;

FIG. 5A is a flow diagram of a processes performed by a processing circuit or a method according to embodiments of the invention;

FIG. 5B shows a set of captured input NIR frames according to an embodiment of the invention;

FIG. 5C shows regions of higher intensity pixels identified in the frames according to an embodiment of the invention;

FIG. 5D shows a key blood flow cluster identified in the frames according to an embodiment of the invention;

FIG. 6A shows two isometric views of an NIR imaged area at different times;

FIG. 6B shows movement of a key blood flow cluster in frames according to an embodiment of the invention;

FIG. 6C illustrates various properties that may be calculated for a blood flow cluster or a key blood flow cluster according to an embodiment of the invention;

FIG. 7 is a flow diagram providing additional details for the steps in the embodiment of FIG. 5A;

FIG. 8 is a flow diagram of steps included in step 502 of FIG. 5A;

FIG. 9 is a flow diagram of steps included in step 702 of FIG. 7;

FIG. 10 is a flow diagram of steps included in step 914 of FIG. 9;

FIG. 11 is a flow diagram of steps included in step 1042 of FIG. 10;

FIG. 12 is a flow diagram of steps included in step 703 of FIG. 7;

FIG. 13A is a flow diagram of steps included in step 1220 of FIG. 12;

FIG. 13B is a flow diagram of steps included in step 1224 of FIG. 12;

FIG. 14 is a flow diagram of steps included in step 1376 of FIG. 13B;

FIG. 15 is a flow diagram of steps included in step 704 of FIG. 7; and

FIG. 16 is a block diagram of a computer or related processing circuitry that implements an embodiment of the invention.

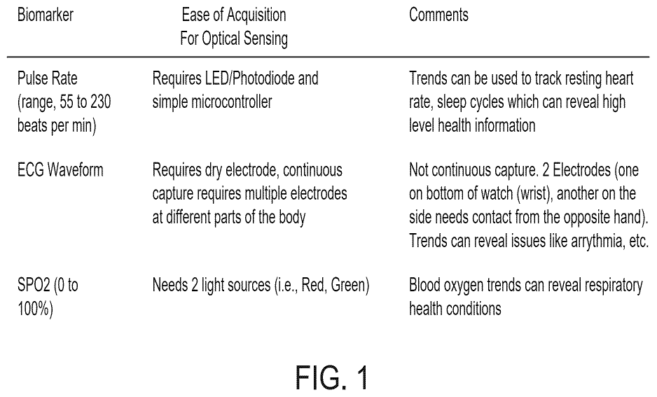

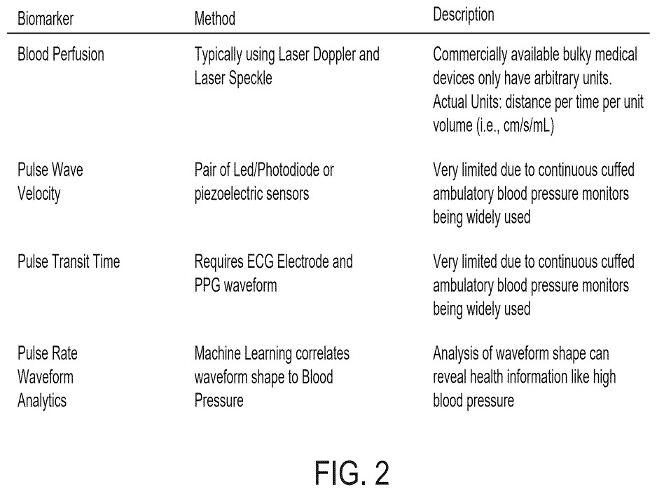

### DETAILED DESCRIPTION

According to an embodiment of the invention, near infrared (NIR) imaging, in contrast to ultrasound and magnetic resonance imaging, may be implemented using inexpensive off-the-shelf components, for example, a CMOS image sensor with sensitivity to the near infrared spectrum, a near infrared light source, and a small computer with minimal specifications that is programmed to calculate blood velocity as discussed further below. According to such an embodiment, contactless near infrared imaging is possible, particularly in a static capture setting like a hospital or lab. According to another embodiment, a contact-type measurement, including a skin conformable setup using large area flexible thin film transistor type sensors (with sensitivity to the near infrared spectrum) can be performed in a portable fashion and in a more active and dynamic setting for remote clinic applications. These embodiments may perform extraction and measurement of blood velocity from high-resolution images with high frame rates, as discussed in further detail below.

Conventional NIR images do not inherently show areas conducive to blood velocity measurements. This presents a challenge for utilizing conventional NIR technology for blood velocity measurement. However, an embodiment of the present invention may advantageously overcome this challenge.

Optical imaging technologies, in contrast to ultrasound and magnetic resonance imaging may be more readily available and cheaper. NIR imaging typically uses a NIR sensitive sensor, and an NIR light source producing light in the 750 nanometers (nm) to 900 nm electromagnetic wavelength spectra, which has the deepest skin penetration (~4 to 5 millimeters (mm) for optical imaging purposes. The NIR imaging is preferably done on areas of the body with shallow or superficial vessels up to 1 centimeter (cm) deep.

Images captured by a near infrared sensor with a near infrared light source illuminating the skin are composed of plurality of pixels that contain digitized data corresponding to the sum of the reflected energy in the area from the layers of the skin that the NIR light penetrates. These layers include the epidermis, the outermost layer around 0.5 mm to 1.5 mm thick, dermis, the capillary containing layer, 1.5 mm to 4 mm thick, and hypodermis also called subcutaneous layer containing the main blood vessel supply along with lipid insulation layer several millimeters thick.

Near infrared light may be reflected by static tissue and fat and absorbed by the hemoglobin present in the various layers, particularly the shallow capillaries in the dermis as well as the arteries and veins in the hypodermis layers. Thus, frames of continuous near infrared sensor images may show the shadow of the veins with a faint pulsing background, where the pulsing is caused by the pressure of the cardiac cycle.

An embodiment of the invention may extract and compute blood velocity biomarker measurements from optical images, particularly of the blood movement in the arteries and veins of the deeper hypodermis layer from the NIR images, in an efficient and cost effective manner.

As mentioned earlier, NIR light is best used to image only certain areas in the human body with superficial or shallow blood vessels that lie within 1 cm from the surface of the skin. Common candidate areas that meet these requirements for most people include the finger, wrist, hand, forearm (palmar side), neck (near the carotid artery), the sides of the forehead, the ankle, and feet.

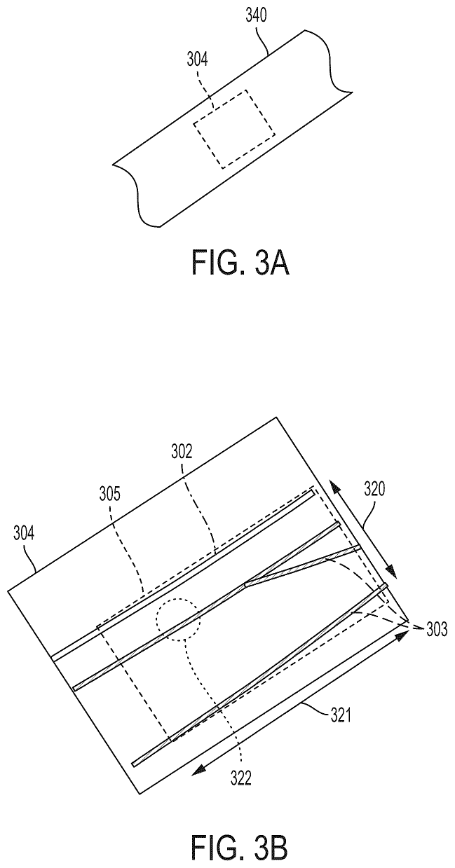

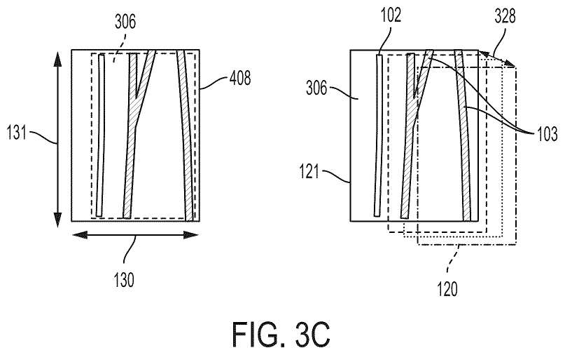

FIG. 3A shows a candidate imaged area 304 on a candidate region 340. The candidate region 340 may be a forearm or any other portion of the subject having superficial blood vessel (i.e., a shallow blood vessel within 5 mm of the skin surface).

FIG. 3B shows the candidate imaged area 304 to be captured by an NIR sensor. The NIR sensor may be pointed towards the candidate region 340, and preferably oriented approximately parallel to a blood vessel in the candidate region 340. In addition, one axis of the candidate imaged area 304 should preferably contain at least three vessels including at least one vein and one artery. Distance 320 is the total distance from the first vessel to the third vessel. The length 321 arranged orthogonally to the distance 320 should be longer than the distance 320 and is preferably at least 1.5 times the distance 320.

First, a candidate imaged area 304 for sensing blood velocity is captured. The candidate imaged area 304 may include a smaller or equally sized imaging area 305 that preferably includes at least three vessels in the width direction 320 including at least one vein and one artery. In the example of FIG. 3B, the candidate imaged area 304 includes an imaging area 305 having one artery 302 and two veins 303. In the example of FIG. 3B, the imaging area 305 includes only three vessels. However, the imaging area may include more than three vessels in the width direction.

FIG. 3C provides an example of a digitized NIR sensed image stream 306 that includes a plurality of images 328 each capturing a view of the imaging area 305, at separate times according to a frame rate. Each image 328 includes at least one artery image 102 of at least one artery in the subject, and at least one vein image 103 of at least one vein in the subject. Each image 328 includes a plurality of pixels arranged in rows r 121 that are arranged along the longest dimension of the frame, and columns c 120 that are arranged along the shortest dimension of the frame. As shown in the example of FIG. 3C, images of arteries 102 may be less easily detected than images of veins 103.

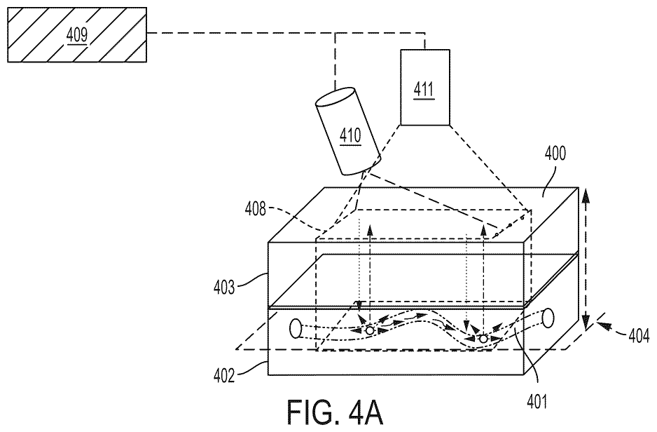

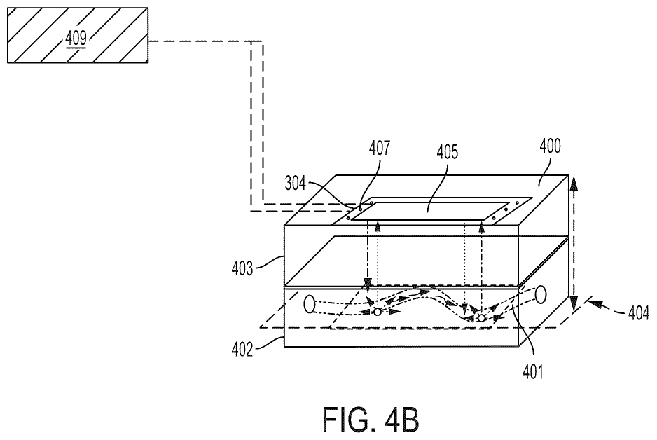

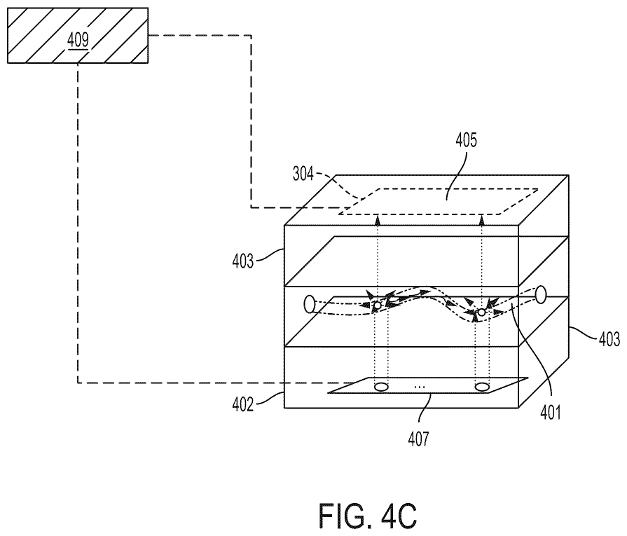

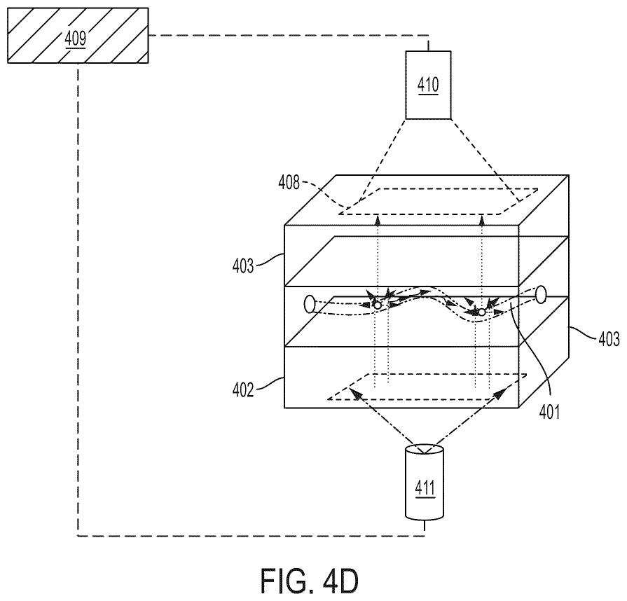

FIG. 4A shows an example according to an embodiment of the invention utilizing a reflective non-contact arrangement. As shown in the embodiment of FIG. 4A, a blood vessel 401 (i.e., an artery or vein) is present in the hypodermis/subcutaneous layer 402, i.e., a layer of the subject's skin that is closest to a NIR penetration depth limit 404 below the epidermis and the dermis layers 403. The embodiment includes an NIR sensor 405 and a non-contact NIR light source 410 that illuminates an imaging area 408. The non-contact NIR sensor 411 according to the embodiment may include a near-infrared sensitive CMOS device, or equivalent and is configured to illuminate the imaging area 408 without requiring contact with the subject. The NIR light source 410 is controlled by a controller 409. The controller 409 receives detected signals from the NIR sensor 411 and performs functions described further below.

FIG. 4B shows a reflective contact-based embodiment using a contacting NIR sensor 405 and a contacting NIR light source 407. Contacting NIR sensor 405 is typically a large area sensor, using thin film transistor type technologies, on a flexible substrate that is configured to be able to remain in contact with the subject. The contacting NIR light source 407 is a light source that may be mounted on the same flexible substrate as the contacting NIR sensor 405 or alternatively mounted on another flexible substrate. This embodiment may be used in dynamic environments where the sensor, light source, and the system would be worn for an extended period of time (e.g., throughout the day). In this example NIR light source 407 lies at the edges of the sensor. However, NIR light source 407 may also be integrated within or behind the sensor pixels or the NIR light source 407 may be omitted entirely, and the NIR light may be provided by the external environment (like the sun's infrared light).

FIG. 4C shows a contact-based transmissive embodiment including a contacting NIR sensor 405 (e.g., a large area skin conformable sensor) arranged on one side of a part of the subject and a flexible contacting NIR light source 407 (e.g., including LED diodes) on the other side. Transmissive contact-based sensor system embodiments may be used in areas where muscle and bone are thin, like fingers, palm, or toes.

FIG. 4D shows an example of a non-contact-based transmissive sensor system embodiment. This uses a non-contacting NIR light source 410 and non-contacting NIR sensor 411. Candidate areas for non-contact transmissive measurement include peripheral areas like the palm or feet where the area is large and where NIR light can transmit through.

In a transmissive embodiment, whether contact based or non-contact, the light source and sensor are placed generally on opposite sides of the subject. The layers between the light source and sensor can then be approximated to contain an epidermis layer 403, a dermis layer, 402, that contains the primary blood vessels, followed by another epidermis layer 403. Although in each embodiment shown above, a dedicated NIR light source is used, the invention also includes embodiments in which no dedicated NIR light source is required, and the NIR sensor captures images using available light. Also, in each “contact” embodiment, at least one of the sensor and light source is in direct contact with a layer of the subject's skin. However, the contact embodiments may also include embodiments in which at least one of the sensor and light source is separated by a small distance (e.g., from microns to a few millimeters) from the subject's skin.

Multiple embodiments discussed above may be used at different candidate sites for one person simultaneously in a high-level system to provide multiple blood velocity biomarkers that maybe used to monitor cardiovascular health trends by tracking the ratio of the blood velocity measurements at these sites.

Thus, an embodiment of the invention may be used to observe effectiveness of therapies applied for addressing various areas of localized pain, such as wrist pain due to carpal tunnel syndrome or migraines. Also, an embodiment of the invention may advantageously be used to track vascular health over time. Portable embodiments of the invention may enable tracking one or more localized medical conditions over time in different environmental situations. For example, an embodiment may simultaneously track vascular health conditions in multiple body parts (i.e., arm, leg, neck, etc . . . ).

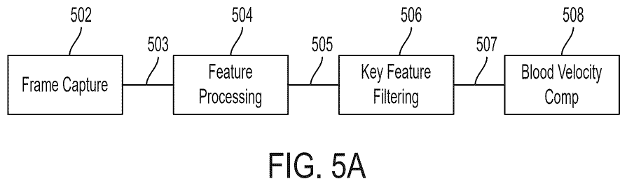

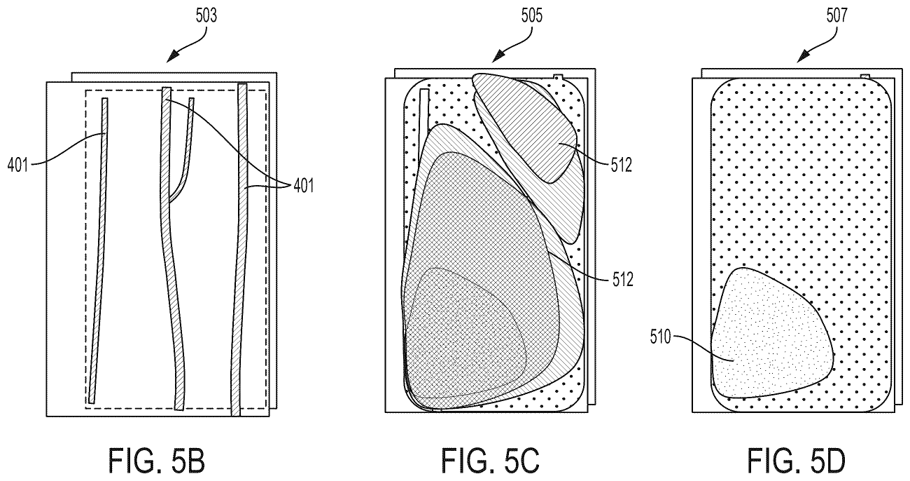

FIG. 5A is a flowchart of an embodiment of a method for quantifying blood velocity measurements from a stream of near infrared sensor input frames. The embodiment includes sensor frame capture 502, feature processing 504, key feature filtering 506, and blood velocity computation 508. Sensor frame capture 502 generates a set of captured sensor input frames 503, for instance in a buffer, and readies them for processing. Feature processing 504 processes the buffered sensor frame set to generate a set of frames 505 within which one or more blood flow clusters are identified.

A blood flow cluster is a region of overlapping blood vessels apparent in the NIR image in which blood appears to be moving in the same speed and direction at a particular time. Each blood flow cluster is a local region of increased blood flow, traveling within the large vessels, medium vessels, and the smaller capillaries, corresponding to a leading edge of a pulse wave. Blood flow clusters can appear in the sensor frames as regions of higher brightness values within a frame.

In feature processing 504, the sensor input frames 503 are evaluated by a filtering processes (e.g., spatial and temporal filters) to include only certain information based on (1) frequencies of interest, and (2) maximum allowed noise per frame. The frequencies of interest may include frequencies close to the cardiac frequency (e.g., 0.9 Hz to 2.5 Hz). The maximum allowed noise is a measure of Signal to Noise Ratio (SNR), typically greater than 40 dB. Thus, the feature processing 504 removes the noise and static information in the image to produce a set of images 505 that more clearly and distinctly reveal the regions of concentrated intensity that move consistently through the images over time.

In the key feature filtering 506 the set of images 505 are first processed to reduce the range of pixel brightness values to isolate lower intensity pixels from higher intensity pixels, and then the regions of higher intensity pixels (i.e., higher brightness pixels). Next in the key feature filtering 506, numerical properties (e.g., area, width, and length) of each higher intensity pixel region is calculated. Next, the regions of higher intensity pixels having properties that meet predetermined requirements are classified as blood flow clusters. Next, the key feature filtering 506 evaluates the changes in properties of the blood flow clusters across consecutive frames over time to identify candidate key blood flow clusters. For example, the movement of the center of mass of each candidate blood flow cluster is evaluated over consecutive frames, and compared to predetermined ranges of allowable movement to identify candidate key blood flow clusters. This step eliminates blood flow clusters that are non-periodic (i.e., not moving in sync with the cardiac rate) and/or inconsistent in their appearance and shape (i.e., shape changes inconsistently between frames). Finally, in key feature filtering 506, frames having multiple candidate key blood flow clusters are ignored to produce a sequence of frames 507 in which each frame has only one key blood flow cluster 510. The key blood flow cluster 510 is identified as one of the blood flow clusters in frames 505 based on relative movement and change of one or more of the blood flow cluster's properties (e.g., movement of center of mass). Blood velocity computation 508 computes blood velocity from the movement of the key blood flow cluster in frames 507.

FIGS. 5B, 5C, and 5D show exemplary frame images at each stage of the method in FIG. 5A. FIG. 5B shows a set of captured sensor input frames 503 produced by sensor frame capture 502. Each of the sensor input frames 503 includes an image of blood vessels 401 that are illuminated and scattered by the NIR light. FIG. 5C shows a set of frames 505 produced by the feature processing 504 in which regions of higher intensity pixels 512 are more clearly depicted. FIG. 5D shows the set of frames 507 produced by feature processing 506. Each of the frames 507 includes only one key blood flow cluster 510. If there are other blood flow clusters in the frames 507, they are ignored.

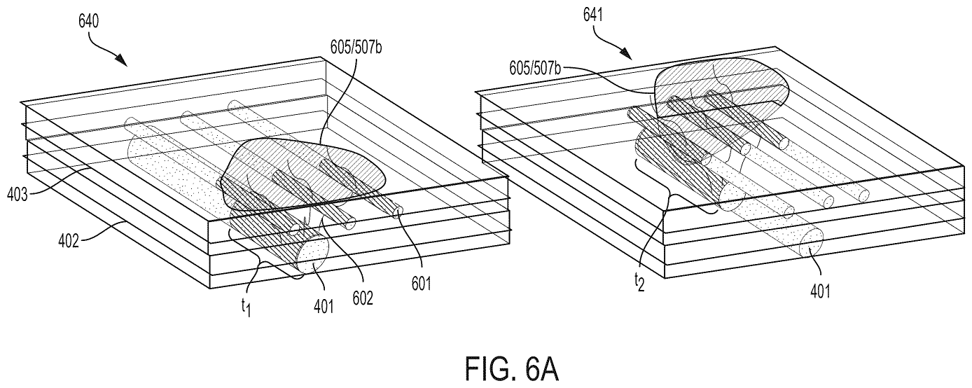

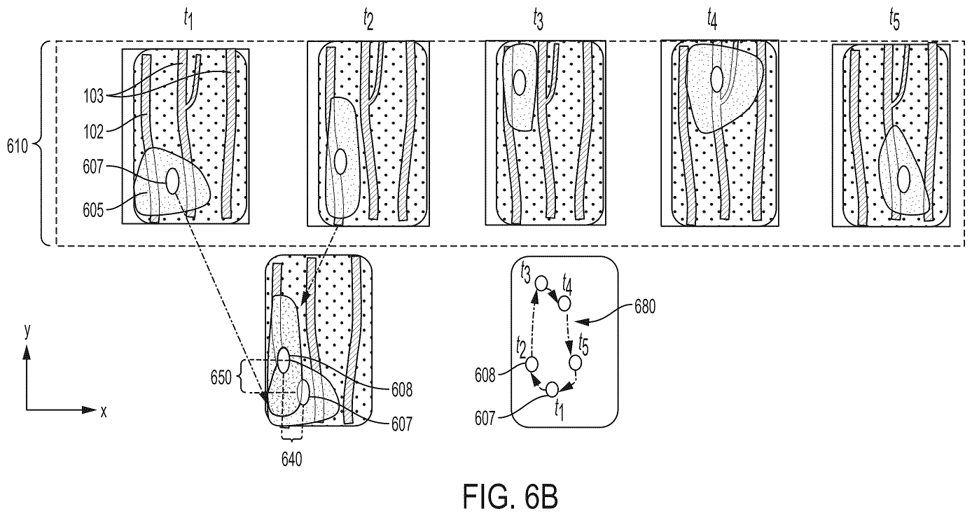

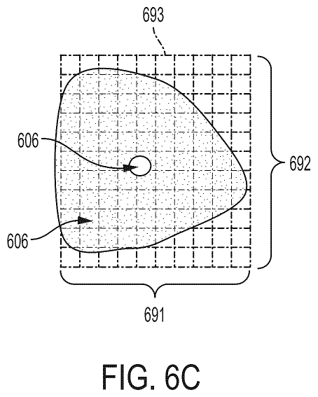

FIG. 6A provides an isometric view of an NIR imaged area of epidermis layer 403 and dermis layer 402 in which a key blood flow cluster 605 has been identified, and illustrates how the key blood flow cluster 605 relates to actual movement of blood in the blood vessel 401. The key blood flow cluster 605 in the set of frames 507 is a two-dimensional view or cross-sectional representation of three-dimensional movement of blood flow to/from the main vessels (arteries, vein) and the surrounding smaller vessels, 601, and capillaries at an instance of time. The key blood flow cluster 605 in a frame, i.e. 693, shows this apparent movement or spread of the blood from the main vessels to an overarching area above the main blood vessel 401 up to the epidermis layer 403 including capillaries 602 and smaller vessels like arterioles or venules 601. Typically, smaller vessels branch off from the primary blood vessel (like the artery or vein) 401, and travel to the layers above. Arterioles or venules 601 are examples of smaller vessels in the dermis layer 402 above the hypodermis layer where the primary blood vessel 401 lies. Region 640 shows a representation of the apparent position of key blood flow cluster 605 at time instance t1, and region 641 shows a representation of the position of the cluster 605 at time instance t2.

FIG. 6B illustrates an example of the movement of a key blood flow cluster in a plurality of frames each including a background of vessels including arteries and veins 102/103 to illustrate that the key blood flow clusters typically move in the lengthwise direction of the blood vessels. In this example, the key blood flow cluster 605 in frame t1 moves along the direction of the reference vessels 102/103 (i.e., up/down direction in the figure). As shown in FIG. 6C, a key blood flow cluster 605 within each frame may contain properties that can be computed using morphological mathematical operations. For example, the properties could include cluster center of mass 606, cluster boundary box x 691, cluster boundary box y 692, and cluster area 693.

The shape as well as other properties of the blood flow cluster may change frame by frame as the blood flow cluster travels across the underlying blood vessels during each cardiac cycle. For example, at time t1, the center of mass coordinate of key blood flow cluster 605 is at location 607, and at time t2, the center of mass coordinate of key blood flow cluster 605 is at location 608. Thus, out of the various computed properties, a center of mass pixel coordinate of the key blood flow cluster 606 may be considered useful in an embodiment of the invention with respect to blood velocity computation. The center of mass coordinates of the key blood flow clusters in the sequence of images at time t1 . . . t5 may be depicted as a cluster movement path 680, which illustrates a path traveled by key blood flow cluster 605 over time during one cardiac cycle. Such a path may be used to validate periodicity of the cluster movement for subsequent frames in the frame set. For example, a sequence of frames containing the key cluster may contain frames captured over several cardiac cycles, where the path of the key blood flow cluster movement may repeat during each of these cardiac cycles. Thus, a key blood flow cluster appears to return to approximately the same position once in each cardiac cycle. The cluster movement path, comprised of a set of x, y pixel coordinates in subsequent cycles maybe compared with each other to ensure repeatability utilizing subsampling and interpolation methods. For instance, each cluster center of mass coordinate maybe stored as a value in an array, wherein one array may contain the cluster center of mass coordinates captured during one cardiac cycle and multiple arrays may contain arrays of the coordinates captured across multiple cardiac cycles.

FIG. 6C illustrates various properties that may be mathematically computed from a region of higher intensity pixels, a blood flow cluster, or a key blood flow cluster (aka, a cluster) within a frame. These are typically in pixel units and may include cluster area, perimeter, x and y axis lengths, and center of mass coordinates. In the time domain, blood flow clusters may be tracked across multiple frames and typically follow the path along its underlying blood vessels every cardiac cycle.

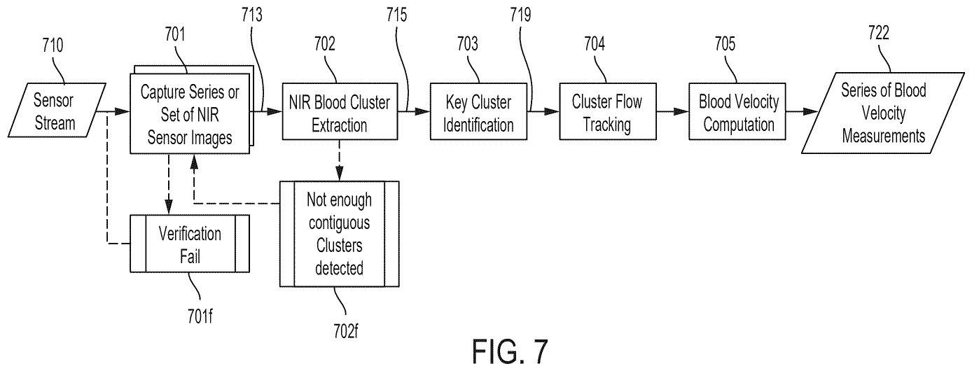

FIG. 7 shows a more detailed example of the embodiment in FIG. 5A. In Step 701 a set or series of NIR frames are captured from a corresponding sensor input stream 710. Step 701 may optionally contain additional steps for efficiency and optimization, including for instance, the identification and validation of temporal and spatial features. This would serve to ensure that only the frames having such features (indicative of proper sensor placement in the candidate areas, frame rate, etc.) are saved for processing in the next steps.

Step 701 produces a buffered output 713, which may contain a validated NIR sensor frame set 713 with the desired spatial and temporal characteristics. In Step 702, blood flow clusters are extracted to produce a potentially validated NIR sensor image set 713. The blood flow clusters contain information pertaining to blood velocity computation. Step 702 may optionally further include additional spatiotemporal filtering and reconstruction steps. If step 702 is successful, frames containing one or more blood flow clusters are output to step 703. However, there may exist a condition, as indicated by step 702f, where a lack of frames containing clusters are identified, and in that case, processing returns to step 701 for recapture.

Step 703 performs a key cluster identification process on the frames containing one or more blood flow clusters to identify exactly one key cluster within all the frames of the frame set containing the one or more clusters. Step 703 produces output 719 including the frame set containing the key cluster.

In Step 704, movements of the key cluster are tracked across all the frames in 719. Step 704 may include the computation of certain properties of the key cluster in each relevant frame, for instance, the centroid property of the key clusters in the frame set. Step 704 outputs data that contains the delta/difference of the cluster movement between consecutive frames in the frame set 719.

Step 705 computes the absolute blood velocity measurements 722, from the output of step 704, with optional filtering to minimize any unnecessary fluctuations. For example, the absolute blood velocity measurements yield results including an amount of blood flow at a current time. Blood velocity measurements are output 722 with units of distance over time (e.g., 2-3 cm/s in a human finger, 5-6 cm/s in a human wrist, or 10-12 cm/s in human arm).

The embodiment may optionally include steps 701f and 702f. Step 701f includes feedback logic to ensure that blood cluster extraction in step 502 is only performed on a validated NIR sensor frame set 713, which is the set of verified frames that have the required spatial and temporal characteristics. If not, then step 701 is performed again to re-capture new sensor frames from the sensor input stream 710 for reprocessing.

Similarly, step 702f includes feedback logic to ensure that valid frames 715, which are output from step 702, contain at least P% of continuous frames compared to the input frame set 713, where all valid frames 715 (aka, reconstructed frame set) contain cluster features which will be used in the next step, step 503. P can be user defined but typically, a valid output set of frames 715 will have a range between a minimum of 70% to maximum of 100% continuous frames of the input frame set 713. For example, an input to 702 may contain an initially validated frame set 713, containing a total of num frames. A one-dimensional average of those frames may contain n PPG (i.e. heart rate or pulse) cycles, where each cycle may contain approximately num/n contiguous frames. Step 702f ensures that a valid frame set output containing cluster features of 702: 1) also contains n pulses, and 2) each pulse contains at least 70% * (num/n) of continuous frames.

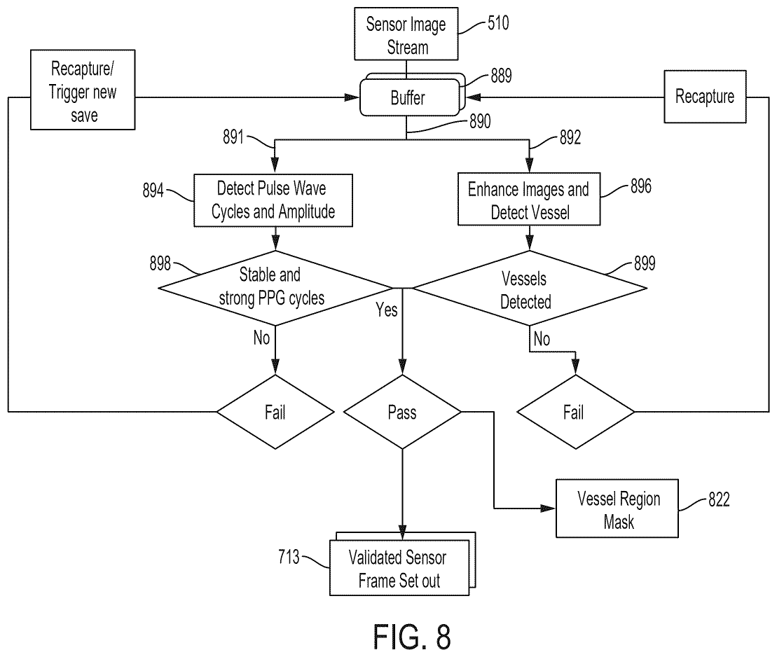

FIG. 8 is a flow diagram of steps included in the step 502 of FIG. 5A. These steps generally involve the capture and validation of a series or set of images from an NIR sensor input stream. The embodiment may contain additional steps to identify and validate an initially captured set for relevant temporal and spatial features (i.e., proper placement in the candidate areas, frame rate).

Step 889 first stores several consecutive frames from an (NIR) input sensor image stream 510 to a buffer or multiple buffers for faster parallel processing as a buffered frame set 890. The buffered frame set 890 may also be processed further parallelly, in branch 891, for temporal validation, and in branch 892, for spatial validation.

In branch 891, an embodiment of the validation may include a step 894 that detects the number of pulse wave cycles and their average peak to peak amplitude within the buffered frame set 890, using for instance a peak detection or similar algorithm. In step 898, the detected pulse wave cycles and their amplitude are compared to predetermined user programmable thresholds for acceptable number of pulse wave cycles and amplitude, after which, a pass/fail decision may be made. For example, at least p pulse wave cycles detection threshold may be set with an average peak to peak amplitude of c counts, where typically, p is at least 3 cycles, and where c maybe set such that 20*log(c/(noise_counts))≥20 db. Noise counts may be set based on the analog front end settings such as amplifier gain, sensor's quiescent noise and the ADCs dynamic range. If step 898 fails, a new frame set is recaptured which then repopulates the buffer 889 with the latest sensor image frames from the sensor image stream 510.

In branch 892, a step 896 performs image enhancement on the buffered frame set 890, for the detection of blood vessels. Examples of vessel filter enhancements may include a version of sato, frangi, or similar filters that highlight or accentuate the vessels within an image.

Following step 896, logic step 899 is performed to decide if valid vessels are detected. For example, if n valid vessels is the validation criteria (where n is typically three), then number of vessels detected in the vessel filtered output of 596 is compared to n for all frames. If step 899 fails, a new frame set is recaptured which then repopulates the buffer 889 with the latest sensor image frames from the sensor image stream 510.

If both logic steps, 898 and 599, of the temporal branch 891 and spatial branch 892, respectively, pass, then output 713 containing the validated sensor frame set is sent to the next step 702 for cluster extraction. Additionally, the binary vessel region mask 822 containing the detected vessel region average may be used in the later stages of this embodiment.

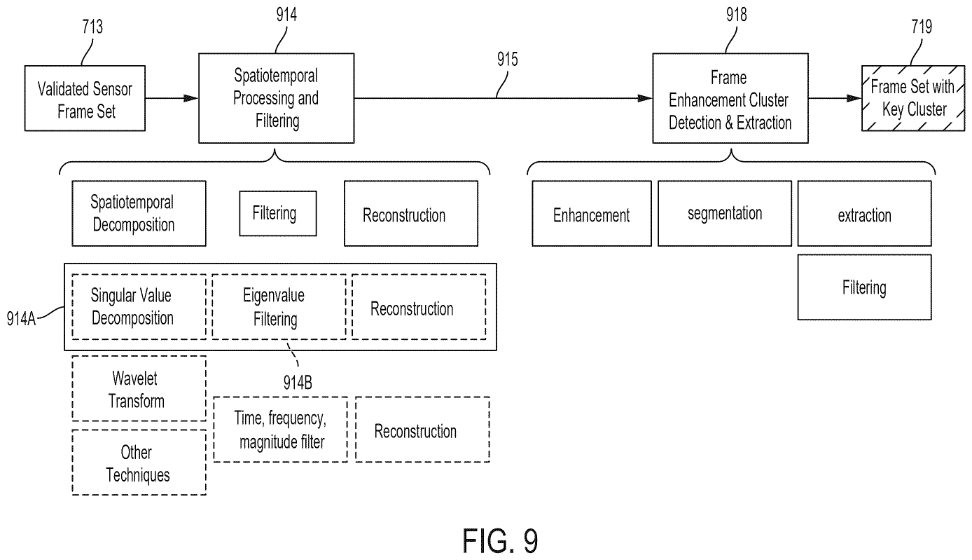

FIG. 9 is a flow diagram of steps included in the step 702 of FIG. 7 that extracts features unique to NIR sensor images needed for blood velocity measurement. In step 914, the validated sensor frame set 713 is first spatiotemporally processed, filtered, and reconstructed to produce reconstructed frames 915. In step 918, feature revealing enhancement may be performed on the reconstructed frames 915.

Step 914 may include one or more of a spatiotemporal decomposition method, subsequent filtering of the resulting decomposition output, and reconstruction. As an example, step 914 may include singular value matrix decomposition 914A, which breaks down a validated frame set 713 (i.e., a three-dimensional matrix array), into smaller arrays, after which eigenvalue filtering 914B can be performed and the filtered constituent arrays can be recombined. The embodiment also includes alternatively applying wavelet transforms or other decomposition techniques, which when combined with a time, frequency, and magnitude filter can also be reconstructed back into the frame set form.

Step 918 may include one or more of feature enhancement operations, feature segmentation operations, feature extraction operations, and filtering operations such that the output of step 918 is a frame set 719 containing only one key cluster.

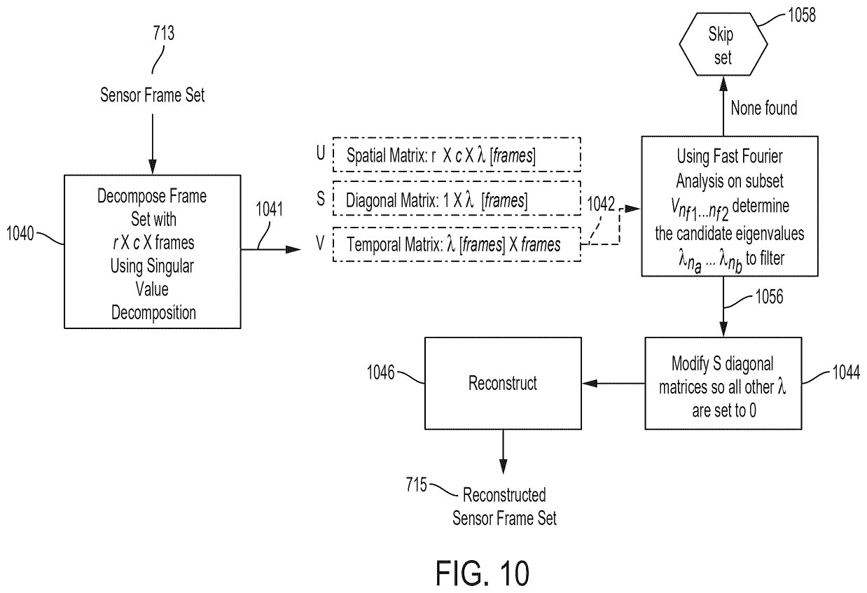

FIG. 10 is a flow diagram of steps included in step 914 of FIG. 9. These steps may be used to process the validated sensor frame set 713 using a type of spatiotemporal processing. In particular, these steps detail a linear algebra matrix array decomposition method called singular value decomposition (SVD). Within these steps is an embodiment of a process for automatically determining the key tuning parameters called eigenvalues for this SVD filter. This automated process is performed in step 1042.

In step 1040, the input sensor frame set 713 is decomposed using SVD to extract the U, S, and V matrices 1041 within it. Each of the U matrices contains image data in the spatial domain, the V matrix contains vector data in the temporal domain, and S matrix is a diagonal matrix containing weights called eigenvalues. The original sensor frame set can be reassembled by multiplying the U, S, and V matrices together. Alternatively, a new reconstructed version of the input frame set can be assembled by changing the values in any of these matrices. Thus, the U, S, and V matrices 1041 have dimensions derived from the input matrices. Spatiotemporally filtering using SVD is typically done by determining which eigenvalues in S matrix to use and setting the rest of the values to 0, which is often done manually by examining the values in U and V. Step 1042 is an automated procedure for determining the candidate eigenvectors necessary for the reconstructed frames to contain the key features. In particular, the filtering process involves determining the useful candidate S matrix values, known as eigenvalues, using the signal magnitude and frequency from the V vectors. There may exist a bypass logic step such that if no candidate eigenvalues are found by step 1042 with the matrices 1041 then the current sensor frame set 713 is skipped. If candidate eigenvalues 1056 are found, then those will be used in step 1044, where the S diagonal matrix is modified such that non-candidate eigenvalues in that matrix are set to 0. In step 1046, the frame set, with the modified S matrix, is reconstructed, by matrix multiplication operation of the U matrix with the modified S matrix before multiplying that with the V matrix.

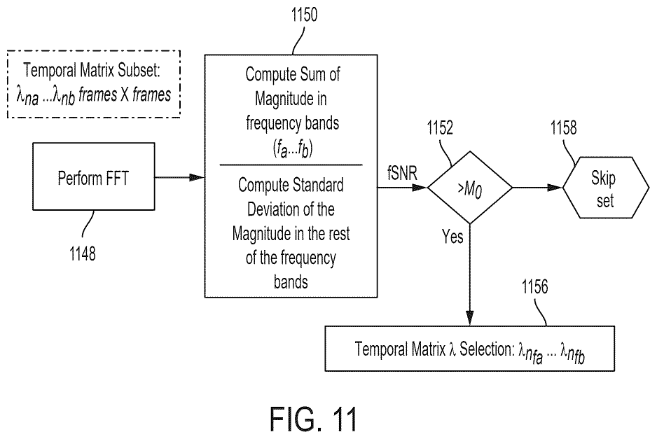

FIG. 11 is a detailed flow diagram of steps performed within step 1042 of FIG. 10. These steps perform automatic spatiotemporal filtering, in particular, as a part of the SVD type operation shown in FIG. 9.

In step 1148, fast Fourier transform (FFT) is performed on a subset of the V temporal matrix. Typically, these are the first few vectors or eigenvectors of V, i.e. V_1 . . . V_20. In step 1150, a frequency Signal to Noise ratio (fSNR) is computed for each of the V matrix subset eigenvectors. Step 1150 includes computing a sum of magnitude in a user defined frequency band fa . . . fb, and dividing that computed sum by a computed standard deviation of the magnitude in the rest of the frequency bands (i.e., the frequency bands outside the range fa . . . fb). Step 1152 checks if each of the subset vectors from the V matrix have a fSNR value that is greater than a predetermined threshold M0, which may indicate if artery/venous periodic information is present. Eigenvalues with qualifying fSNR are output as a temporal matrix 1156.

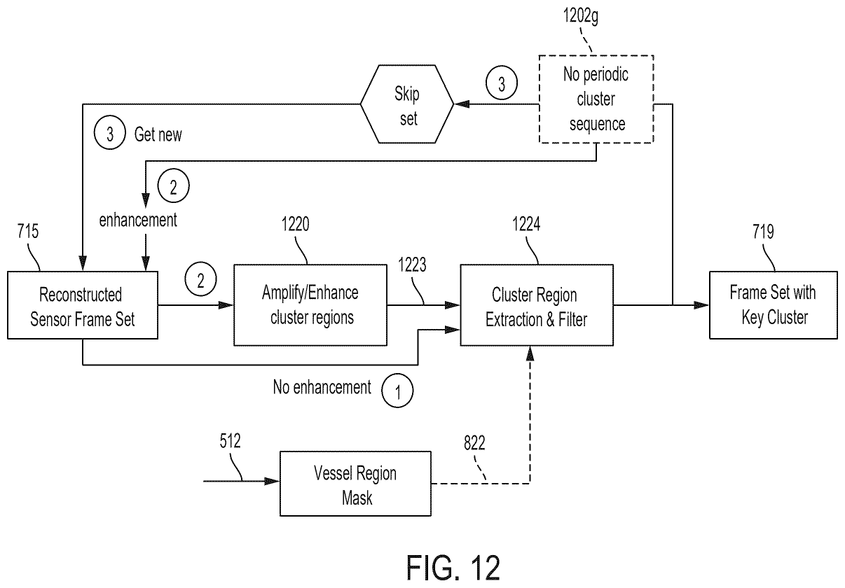

FIG. 12 is a flow diagram of steps that may be included in step 703 of FIG. 7 that perform frame enhancement and cluster detection, extraction, and filtering in order to generate an output frame set 719 with only one key cluster per frame.

This step may be implemented with multiple processing paths to optimize computation time. For example, in path (1), the reconstructed output of the spatiotemporal filtered, sensor frame set 715, which may already contain processable cluster regions and movement, are directly sent to cluster region extraction and filter step 1224, without additional enhancement to save on some processing time.

The cluster region extraction and filter step 1224 performs three main operations: cluster segmentation, property calculation, and filtering. If no periodic cluster sequences are found by the end of step 1224, then frame set 715 is enhanced using step 1220. As shown in path 2, step 1220, may perform dedicated image enhancement operations to further highlight the cluster feature areas with respect to the other areas, before sending the resulting enhanced cluster frames set 1223 to cluster region extraction and filter operation 1224. If there are still no periodic single cluster sequences detected, then as shown in path (3), the current frame set 715 will be skipped and a new set of frames will be processed.

Step 1224, used in both the 1st and 2nd paths, processes each frame in frame set 715 or 1223 respectively, to first determine any segmentable or separable clusters. Next the properties of the clusters with these detectable and segmentable clusters are computed. The properties of the clusters may then be compared with consecutive frames' properties to determine if key clusters exist in frames containing more than one detectable clusters, which eventually dictates whether the bypass logic step 1202g is activated, or if the frame set 719 can be used in the subsequent steps. For example, an enhanced cluster frame set, 1223, or the reconstructed frame set 715, may not only containing frames with just one detectable cluster, but can also contain frames with more than one detectable cluster, and/or no detectable clusters. One of the roles of the filter in step 1224, in this example, is to determine the best cluster on frames containing more than one cluster. For instance, one method could utilize one or more properties, like the center of mass coordinate, area, or the perimeter properties of all clusters in a current frame n, along with the same properties in the previous frame's key cluster (frame n−1). The current frame's key cluster out of more than one detectable cluster may be determined by computing which cluster's properties are the least different from the previous frame (this method is described further in the draft). Additionally, there may be a threshold set to reject the frame set entirely, if there are more than n % of frames containing no usable clusters, that may be defined as unusable if cluster properties in a frame fall outside user defined thresholds.

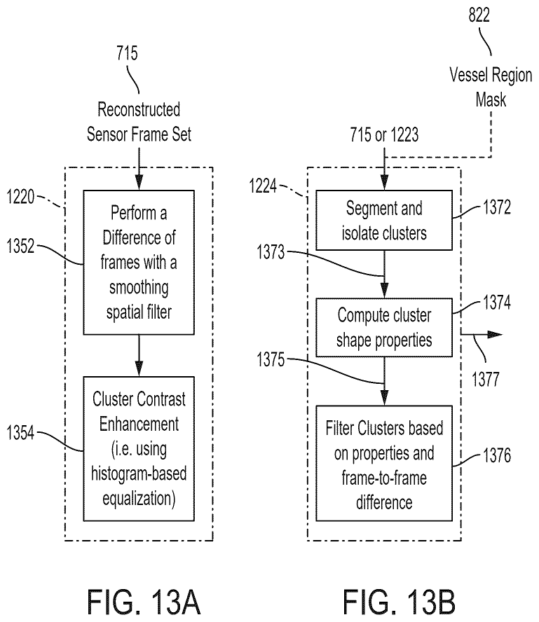

FIG. 13A is a flow diagram steps that may be included within step 1220 of FIG. 12. For example, step 1352 performs a difference of frames operation in which each image frame is subtracted by the subsequent frame to reveal the details changing frame to frame. Step 1352 may also apply a spatial filter, like a median filter or a sliding window average filter, to smooth out random noise that can present itself after the difference operation.

In Step 1354, a cluster specific image enhancement may be performed to further enhance lighter cluster areas using, for example, a histogram-based equalization enhancement method. Step 1354 may auto adjust histogram levels in each image frame to highlight areas where there is constant, periodic change, as these areas represent the areas of blood flow closest to the arterial and venous system.

FIG. 13B shows an embodiment of steps within step 1224 of FIG. 12. Step 1372 performs segmenting and isolating clusters, step 1374 computes cluster properties, and step 1376 filters key clusters based on the difference of the cluster property values. Step 1372 takes each frame of the input set, and separates or segments the cluster regions for each frame based on the intensity, for instance, using existing known methods like k-means clustering. Then using thresholding, a binary mask may be created to enable the separation of the cluster area from the rest of the image in preparation for the next cluster property extraction step. Step 1372 produces output 1373, which is the frame set after segmentation that contains detectable clusters along with a binary mask that isolates the clusters from the rest of the frame. Step 1374 computes cluster properties from post segmentation frame set 1373. The calculated properties may include cluster center of mass location, area, and the cluster bounded box x and y distance. The properties may be extracted using known algorithms/methods such as contour tracing, where the algorithm counts the pixels and the neighboring ones containing similar values.

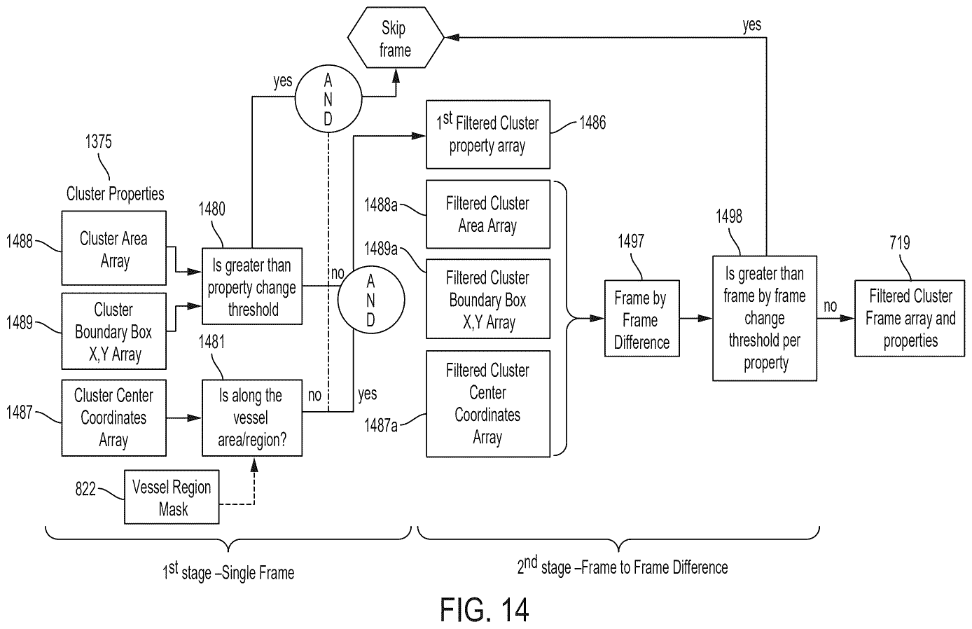

FIG. 14 details an embodiment describing a process within step 1376 of FIG. 13B for a method for identifying a key cluster within frames that may contain two or more clusters per frame. One embodiment may utilize a two-step approach. First, using per frame limits for the frame's cluster's properties, which may also include a mask of the vessel area, 822, to further narrow down on valid cluster locations. Second step may involve, using the difference in the cluster property values between each consecutive cluster containing frame and a respective threshold limit.

The first step to filtering clusters within a frame, may include for example, a logic to ensure that only clusters of a certain area and size can pass, 1480, as well as, 1481, a logic to detect only the clusters with center of mass coordinates along the blood vessel area, to allow for optimal tracking of the cluster center of mass coordinates. For instance, frame 1 may contains n detected clusters with properties, cluster centers of mass 1487, (frame_1 x_c1, frame_1 y_c1), . . . , (frame_1 x_cn, frame_1 y_cn) , cluster areas, 1488, properties frame_1 a_1, . . . , frame_1 a_n, and cluster boundary box width, length, 1489, (frame_1 x_bbox1, frame_1 y_bbox1), . . . , (frame_1 x_bboxn, frame_1 y_bboxn). This filter stage may consider each value in the cluster properties, 1488, 1489, per frame, like frame_1 a_1, . . . , frame_1 a_n to compare with a threshold, 1499, in order to make a decision whether or not to filter a cluster.

Decision thresholds may vary from person to person and candidate measurement sites, but may not surpass user-defined limits such as area_limit<0.25*total pixels, xbbox_limit<0.5 image width, ybbox_limit<0.5 image length.

If any of the cluster property values in a frame is greater than the threshold, in step 1480, then that cluster may be considered as not passing, while the ones below are considered relevant and used in the next filter stage properties step 1486. If all cluster properties in a frame fail, then that frame is not considered relevant and skipped.

The second stage may determine cluster relevancy from frame-to-frame changes of the properties on the frames that have passed the first step serves to eliminate any false cluster movements and jitter. For instance, frame 2's cluster properties, 1487a, 1488a, 1489a, are subtracted from frame 1's and the difference, shown in 1497, is compared with a threshold of step 1498, for each of the properties. If all the three properties satisfy the condition, then frames 2 and 1 are considered passing and saved in the filter cluster array. This is repeated for the properties in each pair of the initially filtered frames in steps 1486. The frames containing passing clusters along with its properties are the frame set 719.

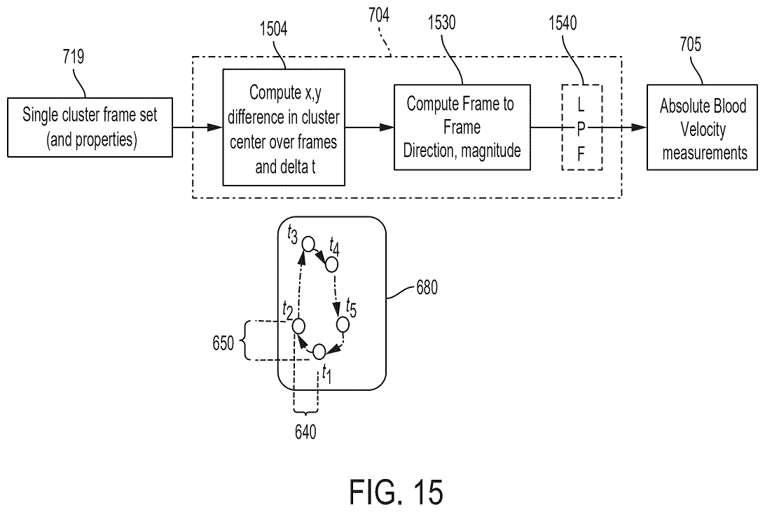

FIG. 15 shows an embodiment of additional features of the key cluster tracking 704 in FIG. 7. The input for step 1504 is the frame set 719 containing a single cluster along with its respective cluster properties. The output of step 705 are a series of blood velocity measurements, typically one for every two frames.

A process of computing blood velocity from frame set 719, the single cluster frame set, may be done, for example, by utilizing the cluster center of mass property. Step 1504 computes the difference in the x and y direction between cluster center of mass coordinates of consecutive frames in that set, which results in an array of delta x and delta y as well as the time difference between the two frames. Frame 680 shows a simple illustration of the process performed in step 704, where the black dots indicate the cluster center of mass coordinates for different consecutive frames, 650 and 640 indicate the change in y and x direction respectively in units of sensor pixels, between cluster center of mass coordinates at times t1 and t2. Delta t is the time difference between t1 and t2, which can be computed by (frame t2−frame t1)/sensor framerate.

Step 1530 may include converting the difference in x and y sensor pixels to physical distance along the subject, using for example, the Euclidian distance formula, and angle by taking the tangent of the two. This provides the initial magnitude and direction of the cluster center of mass movement across the frames. The tangent function and Euclidian distance for example, is shown below:

$$tan ( ( y\_ ( n + 1 ) - y\_n ) / ( x\_ ( n + 1 ) - x\_n ) ) ,$$

$$√ ( ( x\_ ( n + 1 ) - x\_n ) \hat{} 2 + ( y\_ ( n + 1 ) - y\_n ) \hat{} 2 )$$

Step 1540 may include a low pass filter implementation, like a typical Butterworth filter, or Infinite Impulse Filter, that is activated if jitter is detected in the output of step 1530 to enable smoother movement of the blood velocity measurements.

The output of the low pass filter 1540 is converted to absolute blood velocity measurements, with example, SI units of centimeters per second per frame. This conversion is done by taking the absolute value of the magnitude and multiplying by a pixels per centimeter factor based on the sensor resolution and dividing by the time difference between consecutive frames.

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

Methods and systems described herein may be implemented using computer programming or engineering techniques including computer software, firmware, hardware or any combination or subset thereof, wherein the technical effects may include at least processing of data according to the present disclosure.

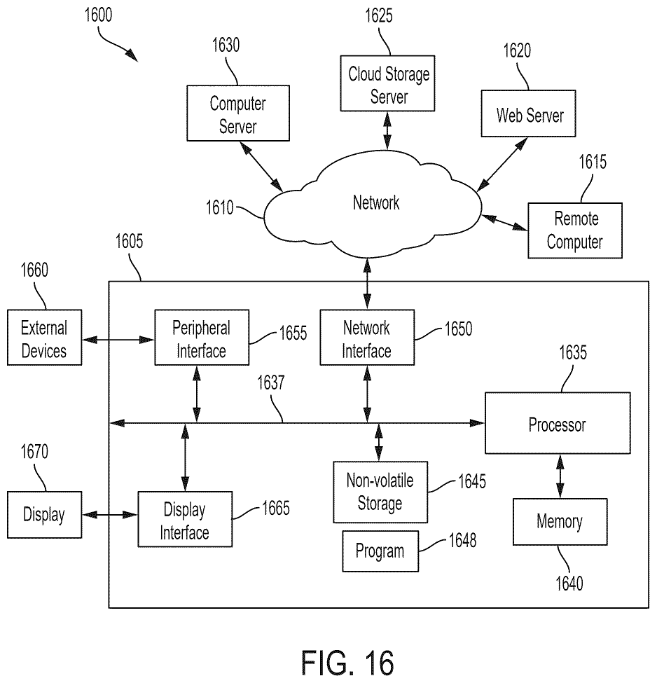

FIG. 16 illustrates a block diagram of a computer or related processing circuitry that may implement at least portions of the various embodiments described herein to perform the steps shown in FIGS. 5 and 7, for example. Control aspects of the present disclosure may be embodied as a system, a method, and/or a computer program product. The computer program product may include a computer readable storage medium on which computer readable program instructions are recorded that may cause one or more processors to carry out aspects of the embodiment.

The computer readable storage medium may be a tangible and non-transitory device that can store instructions for use by an instruction execution device (processor). The computer readable storage medium may be, for example, but is not limited to, an electronic storage device, a magnetic storage device, an optical storage device, an electromagnetic storage device, a semiconductor storage device, or any appropriate combination of these devices. A non-exhaustive list of more specific examples of the computer readable storage medium includes each of the following (and appropriate combinations): flexible disk, hard disk, solid-state drive (SSD), random access memory (RAM), read-only memory (ROM), erasable programmable read-only memory (EPROM or Flash), static random access memory (SRAM), compact disc (CD or CD-ROM), digital versatile disk (DVD), MO, and memory card or stick. A computer readable storage medium, as used in this disclosure, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions implementing the functions described in this disclosure can be downloaded to an appropriate computing or processing device from a computer readable storage medium or to an external computer or external storage device via a global network (i.e., the Internet), a local area network, a wide area network and/or a wireless network. The network may include copper transmission wires, optical communication fibers, wireless transmission, routers, firewalls, switches, gateway computers and/or edge servers. A network adapter card or network interface in each computing or processing device may receive computer readable program instructions from the network and forward the computer readable program instructions for storage in a computer readable storage medium within the computing or processing device.

Computer readable program instructions for carrying out operations of the present disclosure may include machine language instructions and/or microcode, which may be compiled or interpreted from source code written in any combination of one or more programming languages, including assembly language, Basic, Fortran, Java, Python, R, C, C++, C# or similar programming languages. The computer readable program instructions may execute entirely on a user's personal computer, notebook computer, tablet, or smartphone, entirely on a remote computer or computer server, or any combination of these computing devices. The remote computer or computer server may be connected to the user's device or devices through a computer network, including a local area network or a wide area network, or a global network (i.e., the Internet). In some embodiments, electronic circuitry including, for example, programmable logic circuitry, field-programmable gate arrays (FPGA), or programmable logic arrays (PLA) may execute the computer readable program instructions by using information from the computer readable program instructions to configure or customize the electronic circuitry, in order to perform aspects of the present disclosure.

Aspects of the present disclosure are described herein with reference to flow diagrams and block diagrams of methods, apparatus (systems), and computer program products according to embodiments of the disclosure. It will be understood by those skilled in the art that each block of the flow diagrams and block diagrams, and combinations of blocks in the flow diagrams and block diagrams, can be implemented by computer readable program instructions.

The computer readable program instructions that may implement the systems and methods described in this disclosure may be provided to one or more processors (and/or one or more cores within a processor) of a general purpose computer, special purpose computer, or other programmable apparatus to produce a machine, such that the instructions, which execute via the processor of the computer or other programmable apparatus, create a system for implementing the functions specified in the flow diagrams and block diagrams in the present disclosure. These computer readable program instructions may also be stored in a computer readable storage medium that can direct a computer, a programmable apparatus, and/or other devices to function in a particular manner, such that the computer readable storage medium having stored instructions is an article of manufacture including instructions which implement aspects of the functions specified in the flow diagrams and block diagrams in the present disclosure.

The computer readable program instructions may also be loaded onto a computer, other programmable apparatus, or other device to cause a series of operational steps to be performed on the computer, other programmable apparatus or other device to produce a computer implemented process, such that the instructions which execute on the computer, other programmable apparatus, or other device implement the functions specified in the flow diagrams and block diagrams in the present disclosure.

FIG. 16 is a functional block diagram illustrating a networked system 1600 of one or more networked computers and servers. In an embodiment, the hardware and software environment illustrated in FIG. 16 may provide an exemplary platform for implementation of the software and/or methods according to the present disclosure. Referring to FIG. 16, a networked system 1600 may include, but is not limited to, computer 1605, network 1610, remote computer 1615, web server 1620, cloud storage server 1625 and computer server 1630. In some embodiments, multiple instances of one or more of the functional blocks illustrated in FIG. 16 may be employed.

Additional detail of a computer 1605 is also shown in FIG. 16. The functional blocks illustrated within computer 1605 are provided only to establish exemplary functionality and are not intended to be exhaustive. And while details are not provided for remote computer 1615, web server 1620, cloud storage server 1625 and computer server 1630, these other computers and devices may include similar functionality to that shown for computer 1605. Computer 1605 may be a personal computer (PC), a desktop computer, laptop computer, tablet computer, netbook computer, a personal digital assistant (PDA), a smart phone, or any other programmable electronic device capable of communicating with other devices on network 1610.

Computer 1605 may include processor 1635, bus 1637, memory 1640, non-volatile storage 1645, network interface 1650, peripheral interface 1655 and display interface 1665. Each of these functions may be implemented, in some embodiments, as individual electronic subsystems (integrated circuit chip or combination of chips and associated devices), or, in other embodiments, some combination of functions may be implemented on a single chip (sometimes called a system on chip or SoC).

Processor 1635 may be one or more single or multi-chip microprocessors, such as those designed and/or manufactured by Intel Corporation, Advanced Micro Devices, Inc. (AMD), Arm Holdings (Arm), Apple Computer, etc. Examples of microprocessors include Celeron, Pentium, Core i3, Core i5 and Core i7 from Intel Corporation; Opteron, Phenom, Athlon, Turion and Ryzen from AMD; and Cortex-A, Cortex-R and Cortex-M from Arm. Bus 1637 may be a proprietary or industry standard high-speed parallel or serial peripheral interconnect bus, such as ISA, PCI, PCI Express (PCI-e), AGP, and the like.

Memory 1640 and non-volatile storage 1645 may be computer-readable storage media. Memory 1640 may include any suitable volatile storage devices such as Dynamic Random-Access Memory (DRAM) and Static Random-Access Memory (SRAM). Non-volatile storage 1645 may include one or more of the following: flexible disk, hard disk, solid-state drive (SSD), read-only memory (ROM), erasable programmable read-only memory (EPROM or Flash), compact disc (CD or CD-ROM), digital versatile disk (DVD) and memory card or stick.

Program 1648 may be a collection of machine-readable instructions and/or data that is stored in non-volatile storage 1645 and is used to create, manage and control certain software functions that are discussed in detail elsewhere in the present disclosure and illustrated in the drawings. In some embodiments, memory 1640 may be considerably faster than non-volatile storage 1645. In such embodiments, program 1648 may be transferred from non-volatile storage 1645 to memory 1640 prior to execution by processor 1635.

Computer 1605 may be capable of communicating and interacting with other computers via network 1610 through network interface 1650. Network 1610 may be, for example, a local area network (LAN), a wide area network (WAN) such as the Internet, or a combination of the two, and may include wired, wireless, or fiber optic connections. In general, network 1610 can be any combination of connections and protocols that support communications between two or more computers and related devices.

Peripheral interface 1655 may allow for input and output of data with other devices that may be connected locally with computer 1605. For example, peripheral interface 1655 may provide a connection to external devices 1660. External devices 1660 may include devices such as a keyboard, a mouse, a keypad, a touch screen, and/or other suitable input devices. External devices 1660 may also include portable computer-readable storage media such as, for example, thumb drives, portable optical or magnetic disks, and memory cards. Software and data used to practice embodiments of the present disclosure, for example, program 1648, may be stored on such portable computer-readable storage media. In such embodiments, software may be loaded onto non-volatile storage 1645 or, alternatively, directly into memory 1640 via peripheral interface 1655. Peripheral interface 1655 may use an industry standard connection, such as RS-232 or Universal Serial Bus (USB), to connect with external devices 1660.

Display interface 1665 may connect computer 1605 to display 1670. Display 1670 may be used, in some embodiments, to present a command line or graphical user interface to a user of computer 1605. Display interface 1665 may connect to display 1670 using one or more proprietary or industry standard connections, such as VGA, DVI, DisplayPort and HDMI.

As described above, network interface 1650, provides for communications with other computing and storage systems or devices external to computer 1605. Software programs and data discussed herein may be downloaded from, for example, remote computer 1615, web server 1620, cloud storage server 1625 and computer server 1630 to non-volatile storage 1645 through network interface 1650 and network 1610. Furthermore, the systems and methods described in this disclosure may be executed by one or more computers connected to computer 1605 through network interface 1650 and network 1610. For example, in some embodiments the systems and methods described in this disclosure may be executed by remote computer 1615, computer server 1630, or a combination of the interconnected computers on network 1610.

Data, datasets and/or databases employed in embodiments of the systems and methods described in this disclosure may be stored and or downloaded from remote computer 1615, web server 1620, cloud storage server 1625 and computer server 1630.

Numerous modifications and variations of the present invention are possible in light of the above teachings. It is therefore to be understood that within the scope of the appended claims, the invention may be practiced otherwise than as specifically described herein.

What is claimed is:

1. A blood velocity measurement apparatus comprising:
a near-infrared (NIR) light source configured to irradiate a subject with NIR light;
a NIR sensor configured to receive scattered NIR light from the subject and produce a series of NIR image frames, each NIR image frame in the series of NIR image frames representing a two dimensional image of a same region of the subject irradiated by the NIR light source and at a different time; and
processing circuitry configured to calculate a blood velocity value from the series of NIR image frames.

2. The blood velocity measurement apparatus according to claim 1, wherein the blood velocity value includes at least one of an average blood flow velocity and an instantaneous blood flow velocity.

3. The blood velocity measurement apparatus according to claim 1, wherein the processing circuitry is further configured to
identify one or more blood flow clusters within the series of NIR image frames; and
calculate the blood velocity value based on movement the one or more blood flow clusters between frames of the series of NIR image frames.

4. The blood velocity measurement apparatus according to claim 1, wherein the processing circuitry is further configured to:
identify one or more blood flow clusters within the series of NIR image frames;
identify only one of the one or more blood flow clusters as a key blood flow cluster;
determine a center of mass of the key blood flow cluster in the series of NIR image frames;
determine a movement of the key blood flow cluster based on the center of mass of the key blood flow cluster in the series of NIR image frames; and
determine the blood velocity value from the determined movement of the key blood flow cluster in the series of NIR image frames.

5. The blood velocity measurement apparatus according to claim 4, wherein the processing circuitry is further configured to:
identify one or more regions of higher intensity pixels in each frame of the series of NIR image frames;
calculate at least one property of each of the regions of higher intensity pixels; and
identify as the one or more blood flow clusters each region of higher intensity pixels for which the at least one property meets a predetermined requirement.

6. The blood velocity measurement apparatus according to claim 4, wherein the processing circuitry is further configured to determine at least one of the following as the at least one property: a center of mass pixel coordinate of a corresponding region of higher intensity pixels key blood flow, a width of a corresponding region of higher intensity pixels, a length of a corresponding region of higher intensity pixels, and an area of a corresponding region of higher intensity pixels.

7. The blood velocity measurement apparatus according to claim 4, wherein the processing circuitry is further configured to identify one of the one or more blood flow clusters as the key blood flow cluster based on
a degree of similarity of a movement of each blood flow cluster as compared to a cardiac rate of the subject; and
a degree of consistency in a shape of each blood flow cluster from frame to frame in the series of NIR image frames.

8. The blood velocity measurement apparatus according to claim 1, wherein the processing circuitry is further configured to determine the movement of the key blood flow cluster based on a position of a center of mass of the key blood flow cluster in each of a plurality of frames of the series of NIR image frames.

9. The blood velocity measurement apparatus according to claim 1, wherein at least one of the NIR light source and the NIR sensor is not in direct contact with the subject.

10. The blood velocity measurement apparatus according to claim 1, wherein at least one of the NIR light source and the NIR sensor is in direct contact with the subject.

11. The blood velocity measurement apparatus according to claim 1, wherein the NIR light source and the NIR sensor are provided on a same substrate.

12. A method of measuring blood velocity in a subject, the method comprising:
irradiating a subject with NIR light;
receiving scattered NIR light from the subject;
producing a series of NIR image frames, each NIR image frame in the series of NIR image frames representing a two dimensional image of a same region of the subject irradiated by the NIR light and at a different time; and
calculating a blood velocity value from the series of NIR image frames.

13. The method according to claim 12, wherein the blood velocity value includes at least one of an average blood flow velocity and an instantaneous blood flow velocity.

14. The method according to claim 13, further comprising:
identifying one or more blood flow clusters within the series of NIR image frames; and
calculating the blood velocity value based on movement the one or more blood flow clusters between frames of the series of NIR image frames.

15. The method according to claim 12, further comprising:
identifying one or more blood flow clusters within the series of NIR image frames;
identifying only one of the one or more blood flow clusters as a key blood flow cluster;
determining at least one property of the key blood flow cluster in the series of NIR image frames;
determining a movement of the key blood flow cluster based on the at least one property of the key blood flow cluster in the series of NIR image frames; and
determining the blood velocity value from the determined movement of the key blood flow cluster in the series of NIR image frames.

16. The method according to claim 15, further comprising:
identifying one or more regions of higher intensity pixels in each frame of the series of NIR image frames;
calculating at least one property of each of the regions of higher intensity pixels; and
identifying as the one or more blood flow clusters each region of higher intensity pixels for which the at least one property meets a predetermined requirement.

17. The method according to claim 15, further comprising:
determining at least one of the following as the at least one property a center of mass pixel coordinate of a corresponding region of higher intensity pixels key blood flow, a width of a corresponding region of higher intensity pixels, a length of a corresponding region of higher intensity pixels, and an area of a corresponding region of higher intensity pixels.

18. The method according to claim 15, further comprising:
identifying one of the one or more blood flow clusters as the key blood flow cluster based on
a degree of similarity of a movement of each blood flow cluster as compared to a cardiac rate of the subject and
a degree of consistency in a shape of each blood flow cluster from frame to frame in the series of NIR image frames.

19. The method according to claim 12, further comprising:
determining the movement of the key blood flow cluster based on a position of a center of mass of the key blood flow cluster in each of a plurality of frames of the series of NIR image frames.

20. The method according to claim 12, further comprising:
diagnosing a condition of the subject based on the blood velocity value.
